(19) United States
(12) Patent Application Publication
(10) Pub. No.: US 2004/0091877 A1
(43) Pub. Date: May 13, 2004

(54) Method and device for identifying a polymer sequence

(76) Inventors: Wolf Bertling, Erlangen (DE); J?ouml;rg Hassmann, Erlangen (DE); Harald Walter, Erlangen (DE); Thomas Schalkhammer, Kasten (DE); Georg Bauer, Nuremberg (DE)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/333,395

(22) PCT Filed: Jul. 7, 2001
(86) PCT No.: PCT/DE01/02588

(30) Foreign Application Priority Data
Jul. 19, 2000 (DE) 10035451.3

(51) Int. Cl. C12Q 1/68; G01N 33/53

(52) U.S. Cl. 435/6; 435/7.1

(57) ABSTRACT

The invention relates to a method for identifying a first polymer sequence bound to a first phase that reflects electromagnetic waves. The inventive method includes the following steps: a) bringing the first polymer sequence into contact with an affine second polymer sequence, which is directly or indirectly bound, via metallic clusters, to a solid second phase that is permeable to electromagnetic waves; b) radiating electromagnetic waves through the second phase, and; c) detecting the alteration of the properties of the reflected electromagnetic waves.

The invention relates to a method and a device for identifying a first polymer sequence which is bound to a first phase which reflects electromagnetic waves.

WO 98/48275 discloses an optical sensor which can be used to detect nucleic acids, proteins and their ligands. U.S. Pat. No. 5,611,998 discloses an optical sensor which can be used to convert nanometric changes in the thickness of thin films into macroscopic optical signals.

For the detection, the optical sensor is, for example, dipped into a nucleic acid-containing solution. After the sensor has been rinsed and dried, its optical property can be determined. The method using the known sensor requires several steps; it is time-consuming.

The object of the invention is to remove the disadvantages of the prior art. In particular, the intention is to specify a method and a device which can be used to detect biochemical molecules rapidly and simply.

This object is achieved by the features of claims 1 and 17. Expedient further developments of the invention ensue from the features of claims 2 to 16 and 18 to 26.

The invention envisages a method for identifying a first polymer sequence which is bound to a first phase which reflects electromagnetic waves, which method comprises the following steps:

a) bringing the first polymer sequence into contact with a second polymer sequence which has affinity for it and which is bound, directly or indirectly via metallic clusters, to a solid second phase which is permeable for electromagnetic waves,

b) penetrating the second phase with electromagnetic waves, and

c) detecting the change in the properties of the reflected electromagnetic waves.

According to the method according to the invention, the biochemical molecule to be detected does not necessarily have to be present in solution. It can also be bound, for example for labeling purposes, to a solid body, such as a banknote. By simply bringing the second phase, which is permeable for electromagnetic waves, into contact and measuring the optical properties of the reflected light it is possible to immediately determine whether the biomolecule to be detected is bound to the first solid phase. The method can be carried out rapidly and readily.

Advantageously, the electromagnetic waves employed are light, preferably generated by a fluorescent lamp, a light emitting diode (LED), a xenon tube or fluorescent tube, or a laser. The properties of directly reflected or scattered light can be determined particularly readily.

The change in property which is measured can be the absorption in a predetermined spectrum before and/or after the first and the second polymer sequences have been brought into contact. It is furthermore also possible to measure the spectral shift as the change in property, when monochromatic light is used.

In addition, the change in property which is measured can be the time dependent change in absorption and/or reflection during or after the bringing-into-contact and/or separation of the first and second polymer sequences. The change in property can be measured under several angles of incidence which differ from each other. It is also conceivable to measure other changes in the properties of the reflected light. In particular, the choice of which change is detected depends on the particular circumstances of the area of use.

Expediently, the first and second polymer sequences are brought into contact by pressing the first and second phases one on top of the other in the dry. The change in property is expediently detected in dependence on the contact pressure.

In step a, at least one further polymer sequence, which is bound directly, or indirectly via of the metallic clusters, to the second phase, can be brought into contact with the first polymer sequence. This makes it possible to carry out several identification reactions simultaneously.

The first phase, or the first substrate, can be a metal foil on which a, spacing layer which is preferably inert, is expediently applied. It is possible to vary the absorption at particular light wavelengths observed when the phases are pressed on top of each other, by means of the thickness of the spacing layer. In this way, it is possible to preset particular colors as signals.

The spacing layer can be applied in the form of a pattern, preferably of a bar code, onto the first phase and also onto the second phase. The first and/or the second polymer sequence(s) can also be applied to the first and second phases, respectively, in the form of a pattern, preferably of a bar code. The provision of the proposed bar codes is outstandingly suitable for the forgery-proof labeling of banknotes, for example.

For the labeling, either the first phase can be firmly linked to the object to be labeled and, for the detection, the second polymer sequence, which is applied on the second phase, can be brought into contact with the first polymer sequence, which is located on the first phase. However, it is also possible, for the labeling, to firmly link the second phase to the object to be labeled and, for the detection, to bring the first polymer sequence, which is applied on the first phase, into contact with the second polymer sequence, which is located on the second phase.

DNA, RNA, protein, peptide or peptide nucleic acid (PNA), or a structurally related oligomer or polymer, which is formed from one monomer or from different monomers which are coupled in a defined sequence, or a ligand thereof, is expediently used as the first and/or second polymer sequence. Any biochemical molecules possessing selective biorecognitive properties are in principle suitable.

According to the invention, it is envisaged, in a device for identifying a first polymer sequence which is bound to a first phase which reflects electromagnetic waves, that a second phase, which is permeable for electromagnetic waves, possesses, on one surface, a second polymer sequence which is bound directly or indirectly, by way of metallic clusters, such that the second polymer sequence can be brought into contact with the first polymer sequence.

The device according to the invention is suitable, in particular, for use in security and recognition technology; it enables the first polymer sequence to be identified rapidly and simply.

It is not necessary to rinse and dry the device in order to measure the optical properties of the electromagnetic waves which are used.

It has proved to be expedient to produce the metallic clusters from precious metals such as silver, gold or platinum. Metals having good conductivity and corrosion resistance, such as copper, aluminum, zinc or indium, are also suitable. Chemically modified polymer sequences bind particularly well to such metals.

It is possible to use light, preferably generated by a fluorescent lamp, a light emitting diode or a laser, as the electromagnetic waves. Advantageously, the second phase is produced from a material having high surface smoothness, for example glass, or from a flexible, smooth plastic film.

An arrangement for determining the optical properties of the reflected light can be provided as a further component of the device. The arrangement can be used for measuring the absorption in a predetermined spectrum before and/or after the first and second polymer sequences have been brought into contact. In addition, the arrangement can be used to measure the spectral shift of the reflected light. Expediently, the arrangement can be used to measure the optical property under several angles of incidence which differ from each other.

The first and/or second polymer sequence can be DNA, RNA, protein, peptide or peptide nucleic acid, or a structurally related oligomer or polymer, which is composed of different monomers which are coupled in a defined sequence, or a ligand thereof. However, it is also possible to use ss-DNA, ss-RNA or synthetic analogs thereof as the polymer sequence.

In addition to this, polymer sequences composed of identical monomers, what are termed homopolymers, can be used.

In that which follows, the invention is clarified with the aid of the drawing.

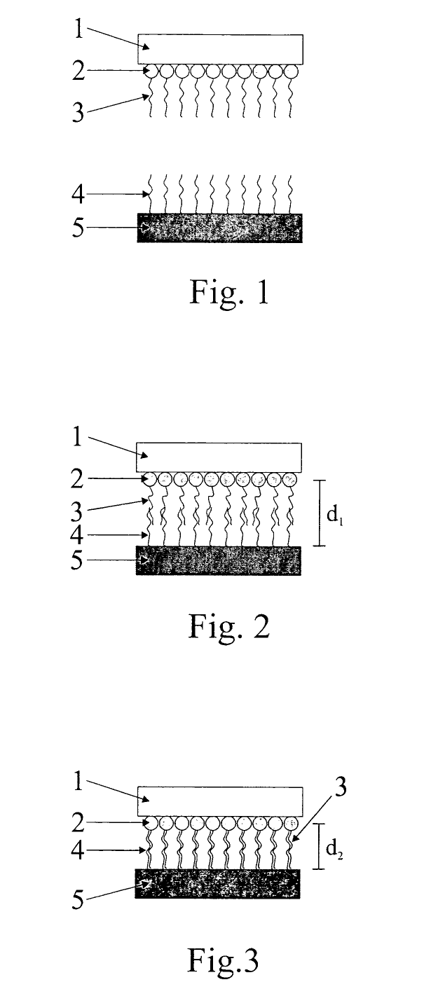

FIG. 1 shows a diagrammatic view of the device,

FIG. 2 shows the device according to FIG. 1 in the case where there is no interaction due to affinity, and

FIG. 3 shows the device according to FIG. 1 in the case where there is interaction due to affinity.

In FIGS. 1-3, a single-stranded DNA 4 is bound, as the first polymer sequence, to a metal foil 5. The metal foil 5 can in turn, for example, be attached, for labeling purposes, to banknotes or chip cards (not depicted here). The second solid phase can, for example, be produced from a glass support 1. Metallic clusters 2, for example gold clusters, are located on one surface of the glass support 1. A further single-stranded DNA 3 is bound, as the second polymer sequence, to the clusters 2.

Provided the DNA 4 and the other DNA 3 are brought into contact, two cases are to be distinguished:

In the first case, shown in FIG. 2, the DNA 4 is not complementary to the other DNA 3. No interaction due to affinity (termed hybridization in the case of DNA) takes place. A first distance d₁ is established between the layer formed by the clusters 2 and the metal foil 5.

In the second case, shown in FIG. 3, the DNA 4 is complementary to the other DNA 3. The DNA 4 and the other DNA 3 hybridize.

A smaller second distance d₂ is established between the layer formed by the clusters 2 and the metal foil 5.

A laser beam (not depicted here) which is incident through the glass support 1 is reflected at the layer which is formed by the clusters 2. The properties of the reflected light depend on the distance d₁, d₂ of the layer formed by the clusters 2 from the metal foil 5. For example, the absorption changes. By measuring the absorption, it can be determined, in a simple manner, whether a specific interaction (in particular hybridization) exists or not. This makes it possible to identify the first polymer sequence.

In order to produce the optical probe designated by the reference numbers 1-3, a glass substrate is, for example, sputter-coated with gold. The DNA, for example oligonucleotides, are in each case provided with a thiol group at their 5′ end. The glass surface, which is sputter-coated with gold, is immersed in a solution containing these oligonucleotides. In connection with this, the oligonucleotides bind to the gold clusters by way of a stable thiol bond.

The sample designated by the reference numbers 4 and 5 is produced in an analogous manner.

The reader is referred to WO 98/48275, whose disclosure content is hereby incorporated by reference, in regard to further details, in particular the sizes of the clusters and the distance parameters. The reader is also referred, in particular, to U.S. Pat. No. 5,611,998, whose disclosure content is hereby incorporated by reference and which describes the change in the spectral properties in dependence on the distances d₁ and d₂, respectively.

What is more, additional security and/or improved signal quality can also be achieved by carrying out the same or other identification reactions on a metal foil which is covered with inert spacing layers of differing thickness.

This makes it possible to read out the identification reactions at different wavelengths. In this connection, the spacing layers can be applied in the form of a bar code pattern or of another pattern.

1. Method for identifying a first polymer sequence (4) which is bound to a first phase (5) which reflects electromagnetic waves, which method comprises the following steps:
a) bringing the first polymer sequence (4) into contact with a second polymer sequence (3) which has affinity for it and which is bound, directly or indirectly via metallic clusters (2), to a solid second phase (1) which is permeable for electromagnetic waves,
b) penetrating the second phase (1) with electromagnetic waves, and
c) detecting the change in the properties of the reflected electromagnetic waves.

2. Method according to claim 1, wherein the electromagnetic waves employed are light, preferably generated by a fluorescent lamp, a xenon lamp, a fluorescent tube, a light emitting diode or a laser.

3. Method according to one of the preceding claims, wherein the change in property which is measured is the absorption in a predetermined spectrum before and/or after the first polymer sequence (4) and the second polymer sequence (3) have been brought into contact.

4. Method according to one of the preceding claims, wherein, when monochromatic light is used, the change which is measured is the spectral shift.

5. Method according to one of the preceding claims, wherein the change in property which is measured is the chronological change in absorption and/or reflection during or after the bringing-into-contact and/or separation of the first polymer sequence (4) and the second polymer sequence (3).

6. Method according to one of the preceding claims, wherein the change in property is measured under several angles of incidence which differ from each other.

7. Method according to one of the preceding claims, wherein the bringing-into-contact is performed by pressing, one on top of the other, in the dry.

8. Method according to one of the preceding claims, wherein the change in property is detected in dependence on the contact pressure.

9. Method according to one of the preceding claims, wherein, in step a, at least one further polymer sequence, which is bound directly, or indirectly by way of the metallic clusters (2), to the second phase (1), is brought into contact with the first polymer sequence (4).

10. Method according to one of the preceding claims, wherein the first phase (5) is a metal foil.

11. Method according to one of the preceding claims, wherein a spacing layer, which is preferably inert, is applied on the metal foil.

12. Method according to one of the preceding claims, wherein the spacing layer is/are [sic] applied in the form of a pattern, preferably of a bar code, onto the first phase (5) or the second phase (1).

13. Method according to one of the preceding claims, wherein the first polymer sequence (4) and/or the second polymer sequence (3) is/are applied in the form of a pattern, preferably of a bar code, onto the first phase (5) and the second phase (1), respectively.

14. Method according to one of the preceding claims, wherein, for the labeling, the first phase (5) is firmly linked to the object to be labeled and, for the detection, the second polymer sequence (3), which is applied on the second phase (1), is brought into contact with the first polymer sequence (4), which is located on the first phase (5).

15. Method according to one of the preceding claims, wherein, for the labeling, the second phase is firmly linked to the object to be labeled and, for the detection, the first polymer sequence (4), which is applied on the first phase (5), is brought into contact with the second polymer sequence (3), which is located on the on the [sic] second phase (1).

16. Method according to one of the preceding claims, wherein DNA, RNA, protein, peptides or peptide nucleic acid (PNA), or a structurally related oligomer or polymer, which is formed from one monomer or from different monomers which are coupled in a defined sequence, or a ligand thereof, is/are used as the first polymer sequence (4) and/or the second polymer sequence (3).

17. Device for identifying a first polymer sequence (4) which is bound to a first phase (5) which reflects electromagnetic waves, wherein a second phase (1), which is permeable for electromagnetic waves, possesses, on one surface, a second polymer sequence (3) which is bound directly or indirectly, by way of metallic clusters (2), such that the second polymer sequence (3) can be brought into contact with the first polymer sequence (4).

18. Device according to claim 17, wherein the metallic clusters (2) are formed from silver, gold, platinum, aluminum, copper, zinc or indium.

19. Device according to claim 17 or 18, wherein the electromagnetic waves employed are light, preferably generated by a fluorescent lamp, a xenon lamp, a fluorescent tube, a light emitting diode or a laser.

20. Device according to one of claims 17 to 19, wherein both phases possess a smooth surface.

21. Device according to one of claims 17 to 20, wherein an arrangement for determining the optical property of the reflected light is provided.

22. Device according to claim 21, wherein the arrangement can be used for measuring the absorption in a predetermined spectral range before and/or after the first polymer sequence (4) and the second polymer sequence (3) have been brought into contact.

23. Device according to claim 21 or 22, wherein the arrangement can be used to measure the spectral shift of the reflected light.

24. Device according to one of claims 21 to 23, wherein the arrangement can be used to measure the optical property under several angles of incidence which differ from each other.

25. Device according to one of claims 17 to 24, wherein the first polymer sequence (4) and/or the second polymer sequence (3) is/are DNA, RNA, protein, peptide or peptide nucleic acid (PNA), or a structurally related oligomer or polymer, which is formed from one monomer or from different monomers which are coupled in a defined sequence, or a ligand thereof.

26. Device according to one of claims 17 to 25, wherein the first polymer sequence (4) and/or the second polymer sequence (3) is/are ss-DNA, ss-RNA, synthetic analogs thereof.
